(19) United States
(12) Patent Application Publication
(10) Pub. No.: US 2016/0303031 A1
(43) Pub. Date: Oct. 20, 2016

(54) SOLID ANHYDROUS COSMETIC COMPOSITION COMPRISING UV-SCREENING AGENTS

(71) Applicant: L'OREAL, Paris (FR)

(72) Inventors: Micheline El ACHKAR, Paris (FR); Audrey RICARD, La Varenne (FR)

(21) Appl. No.: 15/101,229

(22) PCT Filed: Dec. 1, 2014
(86) PCT No.: PCT/EP2014/076039

§ 371 Date: Jun. 2, 2016

(30) Foreign Application Priority Data
Dec. 3, 2013 (FR) 1361978

(51) Int. Cl. A61K 8/891; A61K 8/92; A61K 8/37; A61Q 1/02; A61K 8/29; A61K 8/26; A61K 8/19; A61Q 17/04; A61K 8/02; A61K 8/58

(52) U.S. Cl. CPC A61K 8/891; A61K 8/0241; A61K 8/92; A61K 8/37; A61K 8/585; A61K 8/29; A61K 8/26; A61K 8/19; A61K 8/0216; A61Q 17/04; A61Q 1/02; A61K 2800/31

(57) ABSTRACT

The invention relates to a cosmetic composition comprising at least one liquid fatty phase containing at least one non-volatile oil and
free mineral UV-screening agent particles having a mean elementary size of greater than 0.070 μm;
and at least one mixture of organic UV-screening agents comprising a salicylate UV-screening agent and a cinnamate UV-screening agent.

Use for making up and caring for keratin materials.

One subject of the present invention is a solid anhydrous cosmetic composition comprising in particular a wax, a liquid fatty phase and organic and mineral UV-screening agents. The composition may be a composition for making up or caring for keratin materials such as the skin, eyelids, lips and in particular the skin. Another subject of the invention is a method for making up or caring for keratin materials.

Products for caring for or making up the skin are known in very diverse formulation forms: fluid or thick cream, lotion, stick, cast solid product, loose powder, compact powder.

Some cosmetic products contain UV-screening agents (sunscreens) to protect the skin from the degradation caused by UV-A rays (wavelength between 320 and 400 nm) and UV-B rays (wavelength between 280 and 320 nm).

UV-A rays bring about immediate and persistent tanning of the skin. Daily exposure to UVA rays, even of short duration, under normal conditions can result in damage to the collagen fibres and the elastin, which is reflected by a modification to the microrelief of the skin, the appearance of wrinkles and uneven pigmentation (liver spots, heterogeneity of the complexion).

UV-B rays may bring about a detrimental change in the biomechanical properties of the epidermis, which is reflected by the appearance of wrinkles, leading to premature ageing of the skin.

Protection against UVA and UVB radiation is therefore necessary. An effective photoprotective product must protect against both UVA and UVB radiation.

However, the introduction of certain organic UV-screening agents of a large amount of organic and/or mineral UV-screening agents (more than 5% by weight of the composition) into a composition often leads to stability problems: the UV-screening agent may recrystallize. When the composition is in solid form, cracks may appear at the surface of the solid product, giving it an unattractive appearance. The cosmetic properties of the composition may be impaired, especially due to difficulty in spreading the composition over the skin, causing discomfort on application.

If a large amount of mineral UV-screening agents (titanium dioxide) is incorporated, the product obtained may have a plastery appearance and impart a dry appearance during the application thereof to the skin.

There is therefore a need to have a solid composition that makes it possible to effectively protect the skin against UV radiation while exhibiting good stability properties and good cosmetic properties.

The inventors have demonstrated that by combining, in a solid cosmetic composition, a non-volatile oil, a mixture of two organic UV-screening agents and mineral UV-screening agent particles as defined hereinbelow, it is possible to obtain a composition having a good stability, especially after storing for 2 months at room temperature (25° C.) and at 45° C. (no recrystallization, homogeneous composition). The composition also has good cosmetic properties, especially during the application thereof to keratin materials such as the skin, in particular good spreading. The composition does not have a plastery appearance, in particular after the application thereof to keratin materials. The texture of the composition is not dry, and presents no discomfort after the application thereof to the skin. Furthermore, the composition does not exhibit surface cracking.

Moreover, the composition possesses good photoprotective effectiveness.

The present invention therefore relates to a solid anhydrous cosmetic composition comprising at least one wax, at least one liquid fatty phase containing at least one non-volatile oil and:
free mineral UV-screening agent particles having a mean elementary size of greater than 0.070 μm;
and at least one mixture of organic UV-screening agents comprising a salicylate UV-screening agent and a cinnamate UV-screening agent.

The invention also relates to a method for making up and/or caring for keratin materials, especially the skin or lips, comprising at least one step that consists in applying a composition as defined according to the invention to keratin materials, especially the skin and/or lips.

The invention also relates to a non-therapeutic cosmetic process for limiting the darkening of the skin and/or improving the colour and/or uniformity of the complexion, comprising the application, to the surface of the keratin material, of a composition as defined previously.

The invention also relates to a non-therapeutic cosmetic process for preventing and/or treating the signs of ageing of a keratin material, comprising the application, to the surface of the keratin material, of a composition as defined previously.

The expression “solid composition” is understood to mean a composition that has a hardness such that it does not flow at room temperature under its own weight, especially after 5 minutes, as opposed to compositions referred to as fluid compositions.

Such a composition is advantageously obtained by hot casting and cooling. The cohesion therein is thus ensured by solidification of at least one of its constituents during the implementation.

Cast cosmetic products are particularly appreciated in the field of makeup on account of their ease of use, the infinite number of forms that they can adopt, and their stability associated with their solid nature.

By way of illustration of these cast products, mention may especially be made of the sticks used for making up the lips and the face, in particular the cheekbones or the shadows under the eyes, but also the products cast in small dishes commonly referred to as “hot-cast” products for making up the face or less recently for making up the eyelashes or eyebrows.

Solid products are also advantageous for care products, such as for example lip care sticks.

A composition according to the invention preferably has a hardness greater than or equal to 600 g, in particular between 600 and 1400 g, preferably between 700 and 1300 g and in particular between 800 and 1100 g.

This hardness measurement may be carried out by means of a TA-XT2® texture analyser, sold by the company Rheo.

This measurement is carried out 24 hours after casting the composition in a small metal dish having a capacity of 10 ml, the assembly being maintained at 20° C.

The hardness is likened to the compression force (in grams) measured during the penetration at 20° C. of a P/8 cylinder 8 mm in diameter to a depth of 2 mm and at a speed of 0.5 mm/second with a trigger force of 2 g.

According to one particular embodiment, the composition of the invention has a hardness ranging from 800 to 1100 g measured according to the protocol described above.

For the purposes of the present application, the term “anhydrous composition” is understood to mean a composition comprising less than 5% by weight of water and preferably less than 2% by weight of water, or even free of water, relative to the total weight of the composition. More preferably still, the composition is free of water, the water not being added during the preparation of the composition, but corresponding to the residual water introduced by the mixed ingredients.

The expression “mean particle size” is understood to mean the parameter D[4,3] measured using a “Mastersizer 2000” particle size analyser (Malvern). The light intensity scattered by the particles as a function of the angle at which they are illuminated is converted to size distribution according to the Mie theory. The parameter D[4,3] is measured; this is the mean diameter of the sphere having the same volume as the particle. For a spherical particle, reference will often be made to the “mean diameter”.

The expression “mean elementary size” is understood to mean the size of non-aggregated particles.

The expression “free particles” is understood to mean that the particles present in the composition are not incorporated in a matrix.

Fatty Phase

The composition according to the invention comprises at least one fatty phase, in particular comprising oils and optionally waxes, which may be present in the composition in a content ranging from 60% to 90% by weight, preferably ranging from 70% to 90% by weight, in particular ranging from 75% to 85% by weight, relative to the total weight of said composition.

In particular, a composition of the invention comprises at least one fatty phase that itself comprises at least one oil and at least one wax as mentioned below.

Total Oils

According to one particular embodiment, the fatty phase comprises an oil that may be selected from volatile and non-volatile oils of hydrocarbon, silicone or fluoro type.

The term “oil” means any fatty substance that is in liquid form at room temperature (20-25° C.) and at atmospheric pressure, with the exception of oily UV-screening agents.

The oils may be present in the fatty phase in a content ranging from 40% to 95% by weight, preferably ranging from 50% to 90% by weight and preferentially ranging from 60% to 80% by weight relative to the total weight of said composition.

The oil or oils may in particular be selected from hydrocarbon, silicone or fluoro oils.

The oils may be volatile or non-volatile.

They can be of animal, plant, mineral or synthetic origin.

For the purposes of the present invention, the term “volatile oil” means an oil (or non-aqueous medium) that is capable of evaporating on contact with the skin in less than one hour, at room temperature and at atmospheric pressure. The volatile oil is a volatile cosmetic oil, which is liquid at room temperature, especially having a non-zero vapour pressure, at room temperature and atmospheric pressure, in particular having a vapour pressure ranging from 0.13 Pa to 40 000 Pa (10⁻³ to 300 mmHg), preferably ranging from 1.3 Pa to 13 000 Pa (0.01 to 100 mmHg) and preferentially ranging from 1.3 Pa to 1300 Pa (0.01 to 10 mmHg).

For the purposes of the present invention, the term “non-volatile oil” means an oil with a vapour pressure of less than 0.13 Pa.

For the purpose of the present invention, the term “silicone oil” is understood to mean an oil comprising at least one silicon atom, and in particular at least one Si—O group.

The term “fluoro oil” is intended to mean an oil comprising at least one fluorine atom.

The term “hydrocarbon oil” is intended to mean an oil containing mainly hydrogen and carbon atoms.

The oils may optionally comprise oxygen, nitrogen, sulfur and/or phosphorus atoms, for example in the form of hydroxyl or acid radicals.

Volatile Oils

The volatile oils may be chosen from hydrocarbon oils containing from 8 to 16 carbon atoms, and especially C₈-C₁₆ branched alkanes (also known as isoparaffins), for instance isododecane (also known as 2,2,4,4,6-pentamethylheptane), isodecane and isohexadecane, for instance the oils sold under the trade names Isopar® or Permethyl®.

Volatile oils that may also be used include volatile silicones, for instance volatile linear or cyclic silicone oils, especially those with a viscosity ≦8 centistokes (cSt) (8×10⁻⁶ m²/s) and especially containing from 2 to 10 silicon atoms, and in particular from 2 to 7 silicon atoms, these silicones optionally comprising alkyl or alkoxy groups containing from 1 to 10 carbon atoms. As volatile silicone oils that may be used in the invention, mention may be made in particular of dimethicones with viscosities of 5 and 6 cSt, octamethylcyclo-tetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Volatile fluoro oils such as nonafluoromethoxybutane or perfluoromethylcyclopentane, and mixtures thereof, may also be used.

The volatile oil or oils may be present in a content ranging from 1% to 25% by weight, especially ranging from 5% to 20% by weight and in particular ranging from 10% to 20% by weight relative to the total weight of the fatty phase. These contents take into account the contents of volatile oils introduced by the crosslinked silicone elastomers in the form of gels in volatile oils. According to one particular embodiment, the composition of the invention comprises less than 5% by weight of additional volatile oil, or even less than 1% by weight of additional volatile oil, other than that constituting the carrier of the crosslinked silicone elastomer described below.

The liquid fatty phase comprises at least one non-volatile oil.

Non-Volatile Oils

The non-volatile oils may be chosen especially from non-volatile hydrocarbon, fluoro and/or silicone oils.

Non-volatile hydrocarbon oils that may especially be mentioned include:
hydrocarbon oils of animal origin, such as perhydrosqualene,
hydrocarbon oils of plant origin, such as phytostearyl esters, such as phytostearyl oleate, phytostearyl isostearate and lauroyl/octyldodecyl/phytostearyl glutamate (Ajinomoto, Eldew PS203), triglycerides formed from fatty acid esters of glycerol, in particular in which the fatty acids may have chain lengths ranging from C₄ to C₃₆ and especially from C₁₈ to C₃₆, these oils possibly being linear or branched, and saturated or unsaturated; these oils may especially be heptanoic or octanoic triglycerides, shea oil, alfalfa oil, poppyseed oil, winter squash oil, millet oil, barley oil, quinoa oil, rye oil, candlenut oil, passionflower oil, shea butter, aloe vera oil, sweet almond oil, peach kernel oil, groundnut oil, argan oil, avocado oil, baobab oil, borage oil, broccoli oil, calendula oil, camelina oil, canola oil, carrot oil, safflower oil, hemp oil, rapeseed oil, cottonseed oil, coconut oil, marrow seed oil, wheatgerm oil, jojoba oil, lily oil, macadamia oil, corn oil, meadowfoam oil, St John's Wort oil, monoi oil, hazelnut oil, apricot kernel oil, walnut oil, olive oil, evening primrose oil, palm oil, blackcurrant seed oil, kiwi seed oil, grapeseed oil, pistachio oil, winter squash oil, pumpkin oil, quinoa oil, musk rose oil, sesame oil, soybean oil, sunflower oil, castor oil and watermelon seed oil, and mixtures thereof, or alternatively caprylic/capric acid triglycerides, such as those sold by the company Stéarineries Dubois or those sold under the names Miglyol 810®, 812® and 818® by the company Dynamit Nobel,
linear or branched hydrocarbons, of mineral or synthetic origin, such as liquid paraffins and derivatives thereof, petroleum jelly, polydecenes, polybutenes, hydrogenated polyisobutene such as Parleam, or squalane,
synthetic ethers containing from 10 to 40 carbon atoms,
synthetic esters, for instance oils of formula R₁COOR₂, in which R₁ represents a linear or branched fatty acid residue containing from 1 to 40 carbon atoms, and R₂ represents a hydrocarbon chain that is especially branched, containing from 1 to 40 carbon atoms provided that R₁+R₂≧10. The esters may be chosen especially from fatty acid alcohol esters, for instance cetostearyl octanoate, isopropyl alcohol esters such as isopropyl myristate or isopropyl palmitate, ethyl palmitate, 2-ethylhexyl palmitate, isopropyl stearate, isopropyl isostearate, isostearyl isostearate, octyl stearate, hydroxylated esters, for instance isostearyl lactate, octyl hydroxystearate, diisopropyl adipate, heptanoates, and especially isostearyl heptanoate, alcohol or polyalcohol octanoates, decanoates or ricinoleates, for instance propylene glycol dioctanoate, cetyl octanoate, tridecyl octanoate, 2-ethylhexyl 4-diheptanoate, 2-ethylhexyl palmitate, alkyl benzoate, polyethylene glycol diheptanoate, propylene glycol 2-diethylhexanoate, and mixtures thereof, C₁₂-C₁₅ alcohol benzoates, hexyl laurate, neopentanoic acid esters, for instance isodecyl neopentanoate, isotridecyl neopentanoate, isostearyl neopentanoate, octyldodecyl neopentanoate, isononanoic acid esters, for instance isononyl isononanoate, isotridecyl isononanoate, octyl isononanoate, hydroxylated esters, for instance isostearyl lactate and diisostearyl malate,
polyol esters and pentaerythritol esters, for instance dipentaerythritol tetrahydroxystearate/tetraisostearate,
esters of diol dimers and of diacid dimers, such as Lusplan DD-DA5® and Lusplan DD-DA7® sold by the company Nippon Fine Chemical and described in patent application US 2004/175 338,
copolymers of a diol dimer and of a diacid dimer and esters thereof, such as dilinoleyl diol dimer/dilinoleic dimer copolymers and esters thereof, for instance Plandool-G,
copolymers of polyols and of diacid dimers, and esters thereof, such as Hailuscent ISDA or the dilinoleic acid/butanediol copolymer,
fatty alcohols that are liquid at room temperature, with a branched and/or unsaturated carbon-based chain containing from 12 to 26 carbon atoms, for instance 2-octyldodecanol, isostearyl alcohol, oleyl alcohol, 2-hexyldecanol, 2-butyloctanol and 2-undecylpentadecanol,
C₁₂-C₂₂ higher fatty acids, such as oleic acid, linoleic acid and linolenic acid, and mixtures thereof,
dialkyl carbonates, the two alkyl chains possibly being identical or different, such as dicaprylyl carbonate sold under the name Cetiol CC® by Cognis,
oils of high molar mass, in particular with a molar mass ranging from about 400 to about 10 000 g/mol and in particular from about 650 to about 10 000 g/mol, in particular from about 750 to about 7500 g/mol and more particularly ranging from about 1000 to about 5000 g/mol. As oils of high molar mass that may be used in the present invention, mention may especially be made of oils chosen from:
   lipophilic polymers,
   linear fatty acid esters with a total carbon number ranging from 35 to 70,
   hydroxylated esters,
   aromatic esters,
   C₂₄-C₂₈ branched fatty acid or fatty alcohol esters,
   silicone oils,
   oils of plant origin,
   and mixtures thereof,
fluoro oils that are optionally partially hydrocarbon-based and/or silicone-based, for instance fluorosilicone oils, fluoro polyethers and fluorosilicones as described in the document EP-A-847 752,
silicone oils, for instance linear or cyclic non-volatile polydimethylsiloxanes (PDMSs); polydimethylsiloxanes comprising alkyl, alkoxy or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyldiphenyltrisiloxanes, 2-phenylethyl trimethylsiloxysilicates, and
mixtures thereof.

According to one particular embodiment, the non-volatile oils are chosen from silicone oils, preferably phenyl silicone oils, and hydrocarbon oils, preferably hydrocarbon ester oils of low molecular weight (comprising less than 40 carbon atoms), and advantageously a mixture thereof.

Thus, according to one preferred embodiment, the composition according to the invention comprises at least one phenyl silicone oil and at least one hydrocarbon ester oil.

Advantageously, the total non-volatile oils may be present in a content ranging from 1% to 70% by weight relative to the total weight of the composition, in particular ranging from 25% to 70% by weight, preferably ranging from 40% to 60% by weight, and preferentially ranging from 45% to 55% by weight, relative to the total weight of the composition.

According to one particular embodiment of the invention, the composition comprises at least one hydrocarbon non-volatile oil, in particular at least one low molecular weight hydrocarbon ester oil also known as a “short ester”.

Low Molecular Weight Hydrocarbon Ester Oil

For the purposes of the present invention, the expression “low molecular weight hydrocarbon ester oil” is understood to mean a hydrocarbon ester comprising less than 40 carbon atoms.

Such esters, when they are introduced into the compositions according to the invention, make it possible to further improve the ease of spreading the product over keratin materials.

The esters in accordance with the invention may be monoesters, diesters or polyesters and are more particularly monoesters, i.e. bearing only one ester function. These esters may be linear, branched or cyclic, and saturated or unsaturated. In particular, they are branched and saturated. They may also be volatile or non-volatile.

In particular, the hydrocarbon esters may correspond to the formula RCOOR′ in which RCOO represents a fatty acid residue containing from 2 to 28 carbon atoms, and R′ represents a hydrocarbon chain containing from 1 to 28 carbon atoms. More particularly, the groups R and R′ are such that the corresponding ester is non-volatile.

Advantageously, the mono- , di- or polyester hydrocarbon esters that may be used in the cosmetic compositions in accordance with the invention comprise less than 40 carbon atoms and more than 10 carbon atoms.

These non-volatile esters may especially be C₁₀ to C₂₆ and in particular C₁₄ to C₂₄ esters.

They may be chosen from esters of C₂ to C₁₈ acids and, especially, of C₂ to C₂₀ alcohols or of C₂ to C₈ polyols or mixtures thereof.

They advantageously comprise less than 25 carbon atoms.

According to one particular embodiment, when the hydrocarbon ester comprises less than 25 carbon atoms, it is not a volatile oil.

Thus, the esters may be chosen from a non-limiting list comprising neopentanoic acid esters, for instance isodecyl neopentanoate, isotridecyl neopentanoate, isostearyl neopentanoate and octyldodecyl neopentanoate, isononanoic acid esters, for instance isononyl isononanoate, octyl isononanoate, isodecyl isononanoate, isotridecyl isononanoate and isostearyl isononanoate, but also isopropyl alcohol esters, such as isopropyl myristate, isopropyl palmitate, isopropyl stearate or isostearate, cetyl octanoate, tridecyl octanoate, 2-ethylhexyl 4-diheptanoate and palmitate, alkyl benzoate, polyethylene glycol diheptanoate, and propylene glycol 2-diethylhexanoate, and mixtures thereof. Said ester may also be chosen from synthetic esters, especially of fatty acids, for instance purcellin oil, isopropyl myristate, ethyl palmitate or octyl stearate; hydroxylated esters, for instance isostearyl lactate, octyl hydroxystearate, diisopropyl adipate, and fatty alcohol heptanoates, octanoates or decanoates, and mixtures thereof.

According to one particular embodiment, the short hydrocarbon ester used in the cosmetic composition in accordance with the present invention may be chosen from isononyl isononanoate and 2-ethylhexyl palmitate, and a mixture thereof.

According to one more preferred embodiment, the composition according to the invention comprises at least 2 short esters of different nature, more preferably still a mixture of isononyl isononanoate and 2-ethylhexyl palmitate.

This or these hydrocarbon ester(s) may be used in the composition in a content ranging from 10% to 50% by weight relative to the total weight of the composition, in particular from 20% to 40% by weight, and better still from 25% to 35% by weight, relative to the total weight of the composition.

The composition according to the invention comprises free mineral UV-screening agent particles having a mean elementary size of greater than 0.07 μm.

The mineral UV-screening agent constituting these particles is generally chosen from metal oxides, preferably titanium, zinc or iron oxides or mixtures thereof and more particularly from titanium dioxide and zinc oxide, and mixtures thereof. Particularly preferably, the mineral UV-screening agent is titanium dioxide (TiO₂).

In particular, the titanium dioxide (TiO₂) may be in rutile and/or anatase form and/or in an amorphous form.

The particles of mineral UV-screening agent, in particular of metal oxide, preferably have a mean elementary size generally of between 0.07 and 0.2 μm.

According to one particular embodiment of the invention, the particles may be in powder form.

The particles B of mineral UV-screening agent may be untreated or treated with at least one surface-treatment agent.

Mention may be made, as a non-limiting example of an untreated metal oxide, of untreated titanium dioxide such as the product sold by the company Tayca under the trade name MTY-700B.

The mineral UV-screening agent particles are preferably hydrophobic-modified and may be coated by undergoing one or more surface treatments of chemical, electronic, mechanochemical and/or mechanical nature with compounds as described, for example, in Cosmetics & Toiletries, February 1990, Vol. 105, pp. 53-64, such as amino acids, beeswax, fatty acids, fatty alcohols, anionic surfactants, lecithins, sodium, potassium, zinc, iron or aluminium salts of fatty acids, metal alkoxides (titanium or aluminium alkoxides), polyethylene, silicones, proteins (collagen, elastin), alkanolamines, silicon oxides, metal oxides or sodium hexametaphosphate.

As is known, silicones are organosilicon polymers or oligomers of linear or cyclic, branched or crosslinked structure, of variable molecular weight, obtained by polymerization and/or polycondensation of suitably functionalized silanes, and are essentially constituted of a repetition of main units in which the silicon atoms are linked together via oxygen atoms (siloxane bond), optionally substituted hydrocarbon radicals being directly attached to said silicon atoms via a carbon atom. The term “silicones” also includes the silanes required for their preparation, in particular alkylsilanes.

Preferably, use will be made of metal oxide particles coated with at least one surface-treatment agent selected from a dimethicone or a linear or branched C₁₂-C₁₈ fatty acid and more particularly stearic acid.

The hydrophobic-modified mineral UV-screening agent particles, in particular metal oxide particles, may also be treated with other surface-treatment agents, in particular with cerium oxide, alumina, silica, aluminium compounds such as aluminium hydroxide, or silicon compounds, or mixtures thereof.

Mention may be made, as a non-limiting example of hydrophobic-treated titanium dioxide particles of: the titanium dioxide treated with a dimethicone such as the product sold by the company Tayca under the trade name MTY-700BS; and a titanium dioxide treated with a silicon/alumina/stearic acid or alumina/stearic acid mixture.

According to one particular embodiment of the invention, the mineral UV-screening agent particles may be in the form of an oily dispersion. The oil present in the oily dispersion of mineral UV-screening agent particles is preferably chosen from C₁₂-C₁₅ alkyl benzoates or triglycerides such as caprylic/capric triglyceride.

The oily dispersions of hydrophobic-modified mineral UV-screening agent particles may also comprise at least one dispersant, for instance polyhydroxystearic acid.

Use will be made more particularly of the following oily dispersions of TiO₂ particles treated with a mixture of stearic acid, polyhydroxystearic acid and alumina:
titanium dioxide (and) C₁₂-C₁₅ alkyl benzoate (and) polyhydroxystearic acid (and) stearic acid (and) alumina, for instance the product sold under the trade name Solveil® XT-100 or Solveil® CT-100 by the company Croda;
titanium dioxide (and) caprylic/capric triglyceride (and) polyhydroxystearic acid (and) stearic acid (and) alumina, for instance the product sold under the trade name Solveil® XT-300 by the company Croda.

Preferably, the content of mineral UV-screening agent particles in the composition according to the invention is between 0.1% and 40% by weight, preferably between 0.5% and 30% by weight and more preferably still between 1% and 20% by weight relative to the total weight of the composition.

Organic UV-Screening Agents

The composition according to the invention comprises a mixture of 2 organic UV-screening agents defined above.

The salicylate (or salicylic acid ester) UV-screening agent present in the composition according to the invention may be selected from:

Homosalate (or homomenthyl salicylate) sold under the name “Neo Heliopan HMS PBF” by Symrise or “Eusolex HMS” by Merck/EMD Chemicals,

2-ethylhexyl salicylate sold under the name “Neo Heliopan OS” by Haarmann & Reimer, Dipropylene glycol salicylate sold under the name “Dipsal” by Scher.

Preferably, 2-ethylhexyl salicylate is used.

The salicylate UV-screening agent may be present in the composition according to the invention in a content ranging from 3% to 7% by weight and preferably from 4% to 6% by weight relative to the total weight of the composition.

The cinnamate (or cinnamic acid ester) UV-screening agent may be selected from the compounds corresponding to the following formula (A):

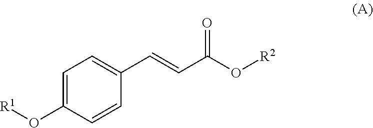

in which:

R¹, R² are, independently of one another, a linear or branched C₁-C₂₄ alkyl radical and more particularly a linear or branched C₁-C₈ alkyl radical, such as methyl, ethyl, propyl, isobutyl, butyl, sec-butyl, isobutyl, pentyl, neopentyl, hexyl and 2-ethylhexyl.

Among the cinnamic acid ester derivatives of formula (A), mention may particularly be made of:
ethylhexyl methoxycinnamate or cinoxate sold in particular under the trade name “Parsol MCX” by DSM,
isopropyl methoxycinnamate,
isoamyl methoxycinnamate sold under the trade name “Neo Heliopan E 1000” by Symrise,
diisopropyl methylcinnamate.

The most preferred being ethylhexyl methoxycinnamate or cinoxate.

The cinnamate UV-screening agent may be present in the composition according to the invention in a content ranging from 3% to 7% by weight and preferably from 4% to 6% by weight relative to the total weight of the composition.

Advantageously, the organic UV-screening agents described above are selected from those that are in the form of oil.

The mixture of UV-screening agents described above is advantageously present in the composition according to the invention in a content ranging from 5% to 20% by weight, preferably ranging from 6% to 15% by weight and preferentially ranging from 8% to 12% by weight relative to the total weight of the composition.

Advantageously, the composition according to the invention contains the mixtures of organic screening agents essentially constituted of:

i) 2-ethylhexyl salicylate

ii) ethylhexyl methoxycinnamate.

Advantageously, the mineral UV-screening agent particles and the 2 salicylate and cinnamate UV-screening agents are present in the composition in a (salicylate UV-screening agent+cinnamate UV-screening agent)/mineral UV-screening agent weight ratio ranging from 1 to 3, preferably ranging from 1.5 to 2.5, and preferentially ranging from 1.8 to 2.2.

The composition according to the invention comprises at least one wax. The wax constitutes a structuring agent of the liquid fatty phase.

For the purposes of the present invention the term “wax” is understood to mean a lipophilic fatty compound, which is solid at room temperature (25° C.) and atmospheric pressure (760 mmHg, i.e. 10⁵ Pa), with a reversible solid/liquid change of state and having in particular a melting point of greater than or equal to 30° C., especially greater than or equal to 55° C. and which may range up to 250° C., especially up to 230° C., and in particular up to 120° C.

By bringing the wax to its melting point, it is possible to make it miscible with oils and to form a microscopically homogeneous mixture, but if the temperature of the mixture is brought back to room temperature, recrystallisation of the wax from the oils of the mixture is obtained.

The melting point values correspond, according to the invention, to the melting peak measured using a differential scanning calorimeter (DSC) such as the calorimeter sold under the name DSC 30® by the company Mettler, with a temperature rise of 5° C. or 10° C. per minute.

The waxes may be present in the composition according to the invention in a content ranging from 0.1% to 15% by weight, especially from 0.1% to 8% by weight, especially from 1% to 8%, in particular from 2% to 6% by weight and better still from 3% to 5% by weight, relative to the total weight of said composition.

The waxes, for the purposes of the invention, may be those used generally in the cosmetic or dermatological fields. They may especially be hydrocarbon, silicone and/or fluoro waxes, optionally comprising ester or hydroxyl functions. They may also be of natural or synthetic origin.

Mention may especially be made, as non-limiting illustrations of these waxes, of:
beeswax, lanolin wax and Chinese insect waxes; rice wax, carnauba wax, candelilla wax, ouricury wax, cork fibre wax, sugar cane wax, Japan wax and sumac wax; montan wax, microcrystalline waxes, paraffin waxes, ozokerites, ceresin wax, lignite wax, polyethylene waxes, the waxes obtained by Fischer-Tropsch synthesis and fatty acid esters and glycerides that are solid at 40° C. and in particular at more than 55° C.,
the waxes obtained by catalytic hydrogenation of animal or vegetable oils having linear or branched C₈-C₃₂ fatty chains, especially hydrogenated jojoba oil, hydrogenated sunflower oil, hydrogenated castor oil, hydrogenated coconut oil and hydrogenated lanolin oil,
silicone waxes or fluoro waxes, and
mixtures thereof.

According to one particular variant of the invention, the wax may be selected from carnauba waxes, microcrystalline waxes and mixtures thereof.

Besides the waxes described above, the compositions according to the invention may also comprise additional fatty substances such as pasty fatty substances.

A composition according to the invention may also comprise at least one pasty compound.

The presence of a pasty compound may make it possible to advantageously confer improved comfort during the deposition of a composition of the invention.

Such a compound may advantageously be selected from lanolin and derivatives thereof; polymeric or non-polymeric silicone compounds; polymeric or non-polymeric fluoro compounds; vinyl polymers, especially homopolymers of olefins; copolymers of olefins; homopolymers and copolymers of hydrogenated dienes; linear or branched oligomers, which are homopolymers or copolymers of alkyl (meth)acrylates preferably containing a C₈-C₃₀ alkyl group; oligomers, which are homopolymers and copolymers of vinyl esters containing C₈-C₃₀ alkyl groups; oligomers, which are homopolymers and copolymers of vinyl ethers containing C₈-C₃₀ alkyl groups; liposoluble polyethers resulting from polyetherification between one or more C₂-C₁₀₀ and in particular C₂-C₅₀ diols; fatty acid or alcohol esters; and mixtures thereof.

Among the esters, mention may be made especially of:
esters of a glycerol oligomer, especially diglycerol esters, such as polyglyceryl-2 triisostearate, condensates of adipic acid and of glycerol, for which some of the hydroxyl groups of the glycerols have reacted with a mixture of fatty acids such as stearic acid, capric acid, stearic acid and isostearic acid, and 12-hydroxystearic acid, like, in particular, those sold under the brand name Softisan 649® by the company Sasol or such as bis-diglyceryl polyacyladipate-2; arachidyl propionate sold under the brand name Waxenol 801® by Alzo; phytosterol esters; fatty acid triglycerides and derivatives thereof, such as hydrogenated coco-glycerides; uncrosslinked polyesters resulting from the polycondensation between a linear or branched C₄-C₅₀ dicarboxylic acid or polycarboxylic acid and a C₂-C₅₀ diol or polyol; aliphatic esters of an ester resulting from the esterification of an aliphatic hydroxycarboxylic acid ester by an aliphatic carboxylic acid; polyesters resulting from the esterification, by a polycarboxylic acid, of an aliphatic hydroxycarboxylic acid ester, said ester comprising at least two hydroxyl groups, such as the products Risocast DA-H® and Risocast DA-L®; and mixtures thereof.

Crosslinked Silicone Elastomers

The composition according to the invention may comprise at least one non-emulsifying crosslinked organopolysiloxane elastomer (also referred to as crosslinked silicone elastomer).

In particular, the non-emulsifying crosslinked organopolysiloxane elastomer used according to the invention is in the form of non-spherical particles.

For the purposes of the present invention, the term “non-emulsifying” silicone elastomer means organopolysiloxane elastomers that do not contain a hydrophilic chain, such as polyoxyalkylene or polyglycerol units.

The non-emulsifying crosslinked silicone elastomer is a crosslinked organopolysiloxane elastomer that may be obtained:
by a crosslinking addition reaction of a diorganopolysiloxane containing at least one hydrogen bonded to silicon and of a diorganopolysiloxane containing ethylenically unsaturated groups bonded to silicon, especially in the presence of a platinum catalyst; or
by a dehydrogenation crosslinking condensation reaction between a hydroxyl-terminated diorganopolysiloxane and a diorganopolysiloxane containing at least one hydrogen bonded to silicon, especially in the presence of an organotin reagent; or
by a crosslinking condensation reaction of a hydroxyl-terminated diorganopolysiloxane and of a hydrolysable organopolysilane; or
by thermal crosslinking of an organopolysiloxane, especially in the presence of an organoperoxide catalyst; or
by crosslinking of an organopolysiloxane with high-energy radiation such as gamma rays, ultraviolet rays or an electron beam.

Preferably, the crosslinked organopolysiloxane elastomer is obtained by a crosslinking addition reaction (A2) of a diorganopolysiloxane containing at least two hydrogens each bonded to a silicon, and (B2) of a diorganopolysiloxane containing at least two ethylenically unsaturated groups bonded to silicon, especially in the presence (C2) of a platinum catalyst, for instance as described in patent application EP-A-295 886.

In particular, the organopolysiloxane may be obtained by reaction of dimethylvinylsiloxy-terminated dimethylpolysiloxane and of trimethylsiloxy-terminated methylhydropolysiloxane, in the presence of a platinum catalyst.

Compound (A2) is the base reactant for the formation of an organopolysiloxane elastomer and the crosslinking takes place via an addition reaction of compound (A2) with compound (B2) in the presence of the catalyst (C2).

Compound (A2) is advantageously a diorganopolysiloxane containing at least two lower (for example C2-C4) alkenyl groups; the lower alkenyl group may be chosen from vinyl, allyl and propenyl groups. These lower alkenyl groups can be located at any position on the organopolysiloxane molecule but are preferably located at the ends of the organopolysiloxane molecule. The organopolysiloxane (A2) may have a branched chain, linear chain, cyclic or network structure, but the linear chain structure is preferred. Compound (A2) may have a viscosity ranging from the liquid state to the gum state. Preferably, compound (A2) has a viscosity of at least 100 centistokes at 25° C.

The organopolysiloxanes (A2) may be chosen from methylvinylsiloxanes, methylvinylsiloxane/dimethylsiloxane copolymers, dimethylvinylsiloxy-terminated dimethylpolysiloxanes, dimethylvinylsiloxy-terminated dimethylsiloxane/methylphenyl-siloxane copolymers, dimethylvinylsiloxy-terminated dimethylsiloxane/diphenyl-siloxane/methylvinylsiloxane copolymers, trimethylsiloxy-terminated dimethylsiloxane/methylvinylsiloxane copolymers, trimethylsiloxy-terminated dimethylsiloxane/methylphenylsiloxane/methylvinylsiloxane copolymers, dimethylvinylsiloxy-terminated methyl(3,3,3-trifluoropropyl)polysiloxanes and dimethylvinylsiloxy-terminated dimethylsiloxane/methyl(3,3,3-trifluoropropyl)siloxane copolymers.

Compound (B2) is in particular an organopolysiloxane containing at least two hydrogens bonded to silicon in each molecule and is thus the crosslinking agent for compound (A2). Advantageously, the sum of the number of ethylenic groups per molecule in compound (A2) and the number of hydrogen atoms bonded to silicon per molecule in compound (B2) is at least 4.

Compound (B2) may be in any molecular structure, especially in a linear chain, branched chain or cyclic structure.

Compound (B2) may have a viscosity at 25° C. ranging from 1 to 50 000 centistokes, especially so as to be well miscible with compound (A).

It is advantageous for compound (B2) to be added in an amount such that the molecular ratio between the total amount of hydrogen atoms bonded to silicon in compound (B2) and the total amount of all the ethylenically unsaturated groups in compound (A2) is in the range from 1/1 to 20/1.

Compound (B2) may be chosen from trimethylsiloxy-terminated methylhydropolysiloxanes, trimethylsiloxy-terminated dimethylsiloxane/methylhydrosiloxane copolymers and dimethylsiloxane/methylhydrosiloxane cyclic copolymers.

Compound (C2) is the catalyst for the crosslinking reaction, and is especially chloroplatinic acid, chloroplatinic acid-olefin complexes, chloroplatinic acid-alkenylsiloxane complexes, chloroplatinic acid-diketone complexes, platinum black and platinum on a support.

The catalyst (C2) is preferably added in an amount of from 0.1 to 1000 parts by weight and better still from 1 to 100 parts by weight, as clean platinum metal, per 1000 parts by weight of the total amount of compounds (A2) and (B2).

Other organic groups may be bonded to silicon in the organopolysiloxanes (A2) and (B2) described previously, for instance alkyl groups such as methyl, ethyl, propyl, butyl or octyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon groups such as an epoxy group, a carboxylate ester group or a mercapto group.

According to one preferred embodiment, the non-emulsifying crosslinked silicone elastomer is generally mixed with at least one hydrocarbon oil and/or silicone oil to form a gel. In these gels, the non-emulsifying elastomer is in the form of non-spherical particles.

According to one particular mode, the non-emulsifying crosslinked silicone elastomer according to the invention is mixed with at least one volatile silicone oil, such as that defined hereinbelow as carrier.

Non-emulsifying elastomers are especially described in patents U.S. Pat. No. 4,970,252, U.S. Pat. No. 4,987,169, U.S. Pat. No. 5,412,004, U.S. Pat. No. 5,654,362, U.S. Pat. No. 5,760,116 U.S. Pat. No. 5,599,533 and U.S. Pat. No. 6,027,738 and in patent application JP-A-61-194 009.

According to one preferred mode, the non-emulsifying crosslinked silicone elastomer used in the present invention may be chosen from Dimethicone Crosspolymers (INCI name) and DimethiconeNinyl Dimethicone Crosspolymers (INCI name).

Non-emulsifying elastomers that may be used include those sold under the names KSG-6, KSG-15, KSG-16, KSG-18, KSG-41, KSG-42, KSG-43, KSG-44, USG-105 and USG-106 by the company Shin-Etsu, DC 9040, DC9041, DC9045, DC 9509, DC9505 and DC 9506 by the company Dow Corning, Gransil by the company Grant Industries, and SFE 839 by the company General Electric.

The non-emulsifying crosslinked silicone elastomer in the form of non-spherical particles is present in the composition in an active material (solids) content ranging from 1% to 6% by weight relative to the total weight of the composition, preferably from 2% to 5% by weight and better still from 3% to 4% by weight relative to the total weight of the composition.

The composition according to the invention may additionally comprise a pulverulent phase.

Pulverulent Phase

The pulverulent phase generally comprises pulverulent fillers and/or colorants.

Thus, the composition according to the invention may comprise at least one filler.

Fillers

The term “fillers” should be understood as meaning colourless or white, mineral or synthetic particles of any form, which are insoluble in the medium of the composition, irrespective of the temperature at which the composition is manufactured.

The fillers may be mineral or organic, of any form, platelet-shaped, spherical or oblong, irrespective of the crystallographic form (for example lamellar, cubic, hexagonal, orthorhombic, etc.). Mention may be made of talc, mica, silica, kaolin, polyamide (Nylon®) powders, poly-β-alanine powder and polyethylene powder, powders of tetrafluoroethylene polymers (Teflon®), lauroyllysine, starch, hollow polymer microspheres such as those of polyvinylidene chloride/acrylonitrile, for instance Expancel® (Nobel Industrie), acrylic acid copolymer microspheres, silicone resin microbeads (for example Tospearls® from Toshiba), polyorganosiloxane elastomer particles, precipitated calcium carbonate, magnesium carbonate, magnesium hydrogen carbonate, hydroxyapatite, barium sulfate, aluminium oxides, polyurethane powders, composite fillers, hollow silica microspheres, glass or ceramic microcapsules, and metal soaps derived from organic carboxylic acids containing from 8 to 22 carbon atoms and preferably from 12 to 18 carbon atoms, for example zinc, magnesium or lithium stearate, zinc laurate or magnesium myristate.

The fillers are preferably chosen from kaolin, talc, silica, hollow polyvinylidene chloride/acrylonitrile microspheres and polyamide powders.

The fillers may be present in the composition in a content ranging from 0.1% to 10% by weight and preferably ranging from 0.1% to 6% by weight relative to the total weight of the composition.

Pigments

The composition according to the invention may comprise a pulverulent colorant, chosen especially from pigments, nacres, interference pigments, glitter and mixtures thereof.

The term “pigments” should be understood as meaning white or coloured, mineral or organic particles of any form, which are insoluble in the physiological medium, and which are intended to colour the composition.

The pigments may be white or coloured, and mineral and/or organic. Among the mineral pigments that may be mentioned are titanium dioxide, optionally surface-treated, zirconium oxide or cerium oxide, and also zinc oxides, iron (black, yellow or red) oxides or chromium oxides, manganese violet, ultramarine blue, chromium hydrate and ferric blue, and metal powders, for instance aluminium powder and copper powder.

The pigments may or may not have undergone a surface treatment dedicated to improving their dispersion and/or to giving them additional properties, for example a hydrophobic nature.

By way of example, the hydrophobic treatment agent may be chosen from fatty acids, such as stearic acid; metal soaps, such as aluminium dimyristate or the aluminium salt of hydrogenated tallow glutamate; perfluoroalkyl phosphates; polyhexafluoropropylene oxides; perfluoropolyethers; amino acids; N-acylamino acids or salts thereof; lecithin; isopropyl triisostearyl titanate; isostearyl sebacate; and mixtures thereof. Preferably, the hydrophobic treatment agent is chosen from fatty acids, amino acids, N-acylamino acids or salts thereof.

The N-acylamino acids may comprise an acyl group having from 8 to 22 carbon atoms, such as, for example, a 2-ethylhexanoyl, caproyl, lauroyl, myristoyl, palmitoyl, stearoyl or cocoyl group. The salts of these compounds may be the aluminium, magnesium, calcium, zirconium, zinc, sodium or potassium salts. The amino acid may be, for example, lysine, glutamic acid or alanine.

Examples of hydrophobic-treated pigments that may be mentioned include the iron oxides and titanium dioxide coated with aluminium stearoyl glutamate sold under the commercial references NAI-TAO-77891®, NAI-C47-051-10®, NAI-C33-8001-10®, NAI-C33-7001-10®, or NAI-C33-9001-10® by the company Miyoshi Kasei.

Among the organic pigments that may be mentioned are carbon black, pigments of D & C type and lakes, especially lakes based on cochineal carmine or on barium, strontium, calcium or aluminium.

The nacres may be chosen from white nacres, such as mica covered with titanium, coloured nacres, such as titanium-coated mica covered with iron oxides, titanium-coated mica covered with in particular ferric blue or with chromium oxide, or titanium-coated mica covered with an organic pigment of the abovementioned type.

Advantageously, the pulverulent colorants are selected from metal oxide pigments.

The pulverulent colorants may be present in the composition according to the invention in a content ranging from 0.1% to 30% by weight, preferably ranging from 2% to 20% by weight and preferentially ranging from 5% to 15% by weight relative to the total weight of the composition.

Other Ingredients

The composition may comprise other ingredients (adjuvants) usually used in cosmetics, such as film-forming polymers, preserving agents, cosmetic active agents, moisturizers, surfactants and fragrances.

Needless to say, a person skilled in the art will take care to select the optional adjuvant(s) added to the composition according to the invention such that the advantageous properties intrinsically associated with the composition in accordance with the invention are not, or are not substantially, adversely affected by the envisaged addition.

The composition may comprise at least one polyol that is miscible with water, in particular at room temperature (25° C.). Mention may be made, as polyols that are miscible with water, of the polyols especially containing from 3 to 20 carbon atoms, preferably containing from 3 to 10 carbon atoms and preferentially containing from 3 to 6 carbon atoms, such as glycerol, propylene glycol, butylene glycol, pentylene glycol, hexylene glycol, dipropylene glycol, diethylene glycol, and mixtures thereof.

The polyol(s) may be present in a content ranging from 5% to 20% by weight, especially ranging from 5% to 17% by weight, preferably of at least 6% by weight, especially ranging from 6% to 20% by weight, preferentially ranging from 6% to 17% by weight and more preferentially ranging from 8% to 12% by weight, with respect to the total weight of the composition.

The composition in cast product form may be prepared by mixing the ingredients of the pulverulent phase and of the fatty phase at high temperature, then by leaving the mixture to cool, in particular in a mould.

This composition may be used in the care and/or makeup field. It may be used as a base, alone, or with a foundation or a powder on top.

The composition according to the invention may be a skin makeup and/or care composition, and may constitute a face powder, an eyeshadow, a face product, a foundation, a concealer product, a body makeup product, a facial care product, a body care product or an antisun product.

The composition of the invention may be used alone or combined with a care and/or makeup product.

According to one particular embodiment, it is used as a makeup base, applied to the skin prior to the application of a makeup composition such as a foundation.

According to another particular embodiment, it is used as a makeup composition as is, applied alone or after application of a makeup base (conventionally a care composition).

The terms “between” and “ranging from” should be understood as including the limits.

The examples that follow are present as nonlimiting illustrations of the field of the invention.

Unless otherwise indicated, the values are indicated as % by weight relative to the total weight of said composition.

### EXAMPLES

### Example 1

### Solid Anhydrous Suntan Composition

The composition containing the following ingredients was prepared:

| | |
|---|---|
| Carnauba wax | 3 |
| Microcrystalline wax (C20-C60) | 5 |
| Microwax HW from | |
| Paramelt | |
| 2-Ethylhexyl palmitate | 10 |
| Isostearyl neopentanoate | 22 |
| Phenyl trimethylsiloxy | 4 |
| trisiloxane (Dow Corning 556 | |
| Cosmetic Grade Fluid from | |
| Dow Corning) | |
| Mixture of crosslinked | 14 |
| polydimethylsiloxane and of | |
| polydimethylsiloxane (6 cSt) | |
| (24/76) (KSG16 from Shin- | |
| Etsu) | |
| 2-Ethylhexyl salicylate | 5 |
| 2-Ethylhexyl 4- | 5 |
| methoxycinnamate | |
| Titanium dioxide surface- | 27 |
| treated by aluminium stearate | |
| and alumina in dispersion in | |
| C12-C15 alkyl benzoate (47% | |
| by weight of TiO₂) (Solaveil | |
| CT-100 from Croda) | |
| Kaolin | 5 |

Preparation

The waxes and the oils were heated at 95° C. then cooled to 85° C. in order to add the crosslinked silicone elastomer.

The UV-screening agents were mixed and heated at 70° C., then added to the preparation.

The pigments and the fillers were added to the preparation and the mixture was milled 3 times using a three-roll mill. The mixture was remelted at 85° C. then cast at 80° C. in small dishes.

The composition obtained has good storage stability after 1 month and after 2 months at room temperature (25° C.) and at 45° C. It applies well to the skin, with good spreading without a feeling of dryness and gives good protection against UV rays.

### Example 2

### Solid Anhydrous Foundation Composition

The composition containing the following ingredients was prepared:

| | |
|---|---|
| Carnauba wax | 3 |
| Microcrystalline wax (C20-C60) | 5 |
| Microwax HW from | |
| Paramelt | |
| 2-Ethylhexyl palmitate | 10 |
| Isostearyl neopentanoate | 22 |
| Phenyl trimethylsiloxy | 4 |
| trisiloxane (Dow Corning 556 | |
| Cosmetic Grade Fluid from | |
| Dow Corning) | |
| Mixture of crosslinked | 14 |
| polydimethylsiloxane and of | |
| polydimethylsiloxane (6 cSt) | |
| (24/76) (KSG16 from Shin- | |
| Etsu) | |
| 2-Ethylhexyl salicylate | 5 |
| 2-Ethylhexyl 4- | 5 |
| methoxycinnamate | |
| Iron oxides | 6 |
| Titanium dioxide surface- | 21 |
| treated by aluminium stearate | |
| and alumina in dispersion in | |
| C12-C15 alkyl benzoate (47% | |
| by weight of TiO₂) (Solaveil | |
| CT-100 from Croda) | |
| Kaolin | 5 |

Composition was prepared according to the method for preparing the composition from Example 1.

The composition obtained has good storage stability after 1 month and after 2 months at room temperature (25° C.) and at 45° C. It applies well to the skin, with good spreading without a feeling of dryness and gives good protection against UV rays.

1. Solid anhydrous cosmetic composition comprising at least one wax and at least one liquid fatty phase containing at least one non-volatile oil and free mineral UV-screening agent particles having a mean elementary size of greater than 0.070 μm;
and at least one mixture of organic UV-screening agents comprising a salicylate UV-screening agent and a cinnamate UV-screening agent.

2. Composition according to claim 1, wherein the mineral screening agent particles are selected from metal oxides.

3. Composition according to claim 1, wherein the mineral UV-screening agent particles are hydrophobic-modified.

4. Composition according to claim 1, wherein the mineral UV-screening agent is present in a content of between 0.1% and 40% by weight relative to the total weight of the composition.

5. Composition according to claim 1, wherein the salicylate UV-screening agent is selected from homomenthyl salicylate, 2-ethylhexyl salicylate and dipropylene glycol salicylate.

6. Composition according to claim 1, wherein the salicylate UV-screening agent is present in a content ranging from 3% to 7% by weight relative to the total weight of the composition.

7. Composition according to claim 1, wherein the cinnamate UV-screening agent is selected from the compounds corresponding to the following formula (A): 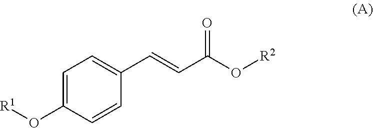
in which:
R¹, R² are, independently of one another, a linear or branched C₁-C₂₄ alkyl radical.

8. Composition according to claim 1, wherein the cinnamate UV-screening agent is selected from ethylhexyl methoxycinnamate, isopropyl methoxycinnamate, isoamyl methoxycinnamate and diisopropyl methylcinnamate.

9. Composition according to claim 1, wherein the cinnamate UV-screening agent is present in a content ranging from 3% to 7% by weight relative to the total weight of the composition.

10. Composition according to claim 1, wherein the non-volatile oil is chosen from silicone oils and hydrocarbon oils, and mixtures thereof.

11. Composition according to claim 1, wherein the non-volatile oil comprises a hydrocarbon ester oil comprising less than 40 carbon atoms.

12. Composition according to claim 1, wherein the content of total non-volatile oils ranges from 1% to 70% by weight relative to the total weight of the composition.

13. Composition according to claim 1, which comprises a crosslinked silicone elastomer.

14. Composition according to claim 1, which comprises at least one filler.

15. Composition according to claim 1, wherein the wax is present in a content ranging from 1% to 8% by weight relative to the total weight of said composition.

16. Composition according to claim 1, which comprises pulverulent colorants.

17. Composition according to claim 1, wherein the wax is chosen from:
beeswax, lanolin wax and Chinese insect waxes; rice wax, carnauba wax, candelilla wax, ouricury wax, cork fibre wax, sugar cane wax, Japan wax and sumac wax; montan wax, microcrystalline waxes, paraffin waxes, ozokerites, ceresin wax, lignite wax, polyethylene waxes, the waxes obtained by Fischer-Tropsch synthesis and fatty acid esters and glycerides that are solid at 40° C.,
the waxes obtained by catalytic hydrogenation of animal or vegetable oils having linear or branched C₈-C₃₂ fatty chains,
silicone waxes or fluorinated waxes, and
mixtures thereof.

18. Composition according to claim 1, which constitutes a face powder, an eyeshadow, a face product, a foundation, a concealer product, a body makeup product, a facial care product, a body care product or an antisun product.

19. Method for making up and/or caring for the skin, comprising at least one step that consists in applying, to said skin, a composition as defined according to claim 1.

20. Composition according to claim 1, wherein the mineral screening agent particles are selected from titanium oxides or zinc oxides and mixtures thereof.
